(19) United States
(12) Patent Application Publication
(10) Pub. No.: US 2012/0078524 A1
(43) Pub. Date: Mar. 29, 2012

(54) System and method for diagnosis tissue samples using fluorescence and raman techniques

(75) Inventors: Shona Stewart, Pittsburgh, PA (US); Jeffrey Cohen, Pittsburgh, PA (US); Amy Drauch, Carnegie, PA (US); John Maier, Pittsburgh, PA (US)

(73) Assignee: Chemlmage Corporation, Pittsburgh, PA (US)

(21) Appl. No.: 13/136,366

(22) Filed: Jul. 29, 2011

Related U.S. Application Data
(63) Continuation-in-part of application No. 12/834,370, filed on Jul. 12, 2010, now Pat. No. 7,990,533.
Application No. 12/834,370 is a continuation-in-part of application No. 12/188,796, filed on Aug. 8, 2008, now Pat. No. 8,013,991.
(60) Provisional application No. 60/954,607, filed on Aug. 8, 2007.

(51) Int. Cl. G01N 33/48; G01N 21/64; G01J 3/44

(52) U.S. Cl. 702/19; 356/72; 356/301

(57) ABSTRACT

A system and method for determining a diagnosis of a test biological sample. A system comprising a first illumination source to illuminate a sample, a first detector for generating a fluorescence data set of said sample, a means for determining a region of interest, a second illumination source to illuminate said region of interest, a second detector to generate a Raman data set of said region of interest, and a means for determining a diagnosis of said sample. A method comprising illuminating a sample, generating a fluorescence data set of said sample, and assessing the fluorescence data set to identify a region of interest, illuminating a region of interest, and generating Raman data set. This Raman data set may be assessed to determine a diagnosis of the sample. A diagnosis may include a metabolic state, a clinical outcome, a disease progression, a disease state, and combinations thereof.

### RELATED APPLICATIONS

This Application is a continuation-in-part of pending U.S. patent application Ser. No. 12/834,370, filed on Jul. 12, 2010, entitled, “System and Method for Analyzing Biological Samples Using Raman Molecular Imaging,” which itself claims priority to U.S. Pat. No. 7,755,757, filed on Sep. 8, 2008, entitled “Distinguishing Between Renal Oncocytoma And Chromophobe Renal Cell Carcinoma Using Raman Molecular Imaging” and U.S. Pat. No. 7,808,633, filed on Feb. 14, 2008, entitled “Spectroscopic System And Method For Predicting Outcome Of Disease.” This Application is also a continuation-in-part of pending U.S. patent application Ser. No. 12/188,796, filed on Aug. 8, 2008, entitled Raman Difference Spectra Based Classification,” which claims priority under 35 U.S.C. §119(e) to U.S. Provisional Patent Application No. 60/954,607, filed on Aug. 8, 2007, entitled “Gleason Score Based Cancer Tissue Analysis.” These applications are hereby incorporated by reference in their entireties.

### BACKGROUND

The biochemical composition of a cell is a complex mix of biological molecules including, but not limited to, proteins, nucleic acids, lipids, and carbohydrates. The composition and interaction of the biological molecules determines the metabolic state of a cell. The metabolic state of the cell will dictate the type of cell and its function (i.e., red blood cell, epithelial cell, etc.). Tissue is generally understood to mean a group of cells that work together to perform a function. Spectroscopic techniques provide information about the biological molecules contained in cells and tissues and therefore provide information about the metabolic state. As the cell's or tissue's metabolic state changes from the normal state to a diseased state, spectroscopic techniques can provide information to indicate the metabolic change and therefore serve to diagnose and predict the outcome of a disease. Cancer is a prevalent disease, so physicians are very concerned with being able to accurately diagnose cancer and to determine the best course of treatment.

Spectroscopic imaging combines digital imaging and molecular spectroscopy techniques, which can include Raman scattering, fluorescence, photoluminescence, ultraviolet, visible and infrared absorption spectroscopies. When applied to the chemical analysis of materials, spectroscopic imaging is commonly referred to as chemical imaging. Instruments for performing spectroscopic (i.e. chemical) imaging typically comprise an illumination source, image gathering optics, focal plane array imaging detectors and imaging spectrometers.

In general, the sample size determines the choice of image gathering optic. For example, a microscope is typically employed for the analysis of sub micron to millimeter spatial dimension samples. For larger objects, in the range of millimeter to meter dimensions, macro lens optics are appropriate. For samples located within relatively inaccessible environments, flexible fiberscope or rigid borescopes can be employed. For very large scale objects, such as planetary objects, telescopes are appropriate image gathering optics.

For detection of images formed by the various optical systems, two-dimensional, imaging focal plane array (FPA) detectors are typically employed. The choice of FPA detector is governed by the spectroscopic technique employed to characterize the sample of interest. For example, silicon (Si) charge-coupled device (CCD) detectors or CMOS detectors are typically employed with visible wavelength fluorescence and Raman spectroscopic imaging systems, while indium gallium arsenide (InGaAs) FPA detectors are typically employed with near-infrared spectroscopic imaging systems.

Spectroscopic imaging of a sample can be implemented by one of two methods. First, a point-source illumination can be provided on the sample to measure the spectra at each point of the illuminated area. Second, spectra can be collected over the an entire area encompassing the sample simultaneously using an electronically tunable optical imaging filter such as an acousto-optic tunable filter (AOTF), a multi-conjugate tunable filter (MCF), or a liquid crystal tunable filter (LCTF). Here, the organic material in such optical filters are actively aligned by applied voltages to produce the desired bandpass and transmission function. The spectra obtained for each pixel of such an image thereby forms a complex data set referred to as a hyperspectral image which contains the intensity values at numerous wavelengths or the wavelength dependence of each pixel element in this image.

The ability to determine a disease state is critical to histological analysis. Such testing often requires obtaining the spectrum of a sample at different wavelengths. Conventional spectroscopic devices operate over a limited range of wavelengths due to the operation ranges of the detectors or tunable filters possible. This enables analysis in the Ultraviolet (UV), visible (VIS), near infrared (NIR), short wave infrared (SWIR) mid infrared (MIR) wavelengths and to some overlapping ranges. These correspond to wavelengths of about 180-380 nm (UV), 380-700 nm (VIS), 700-2500 nm (NIR), 850-1700 nm (SWIR) and 2500-25000 nm (MIR).

Various types of spectroscopy and imaging may be explored for detection of various types of diseases in particular cancers. Raman spectroscopy is based on irradiation of a sample and detection of scattered radiation, and it can be employed non-invasively to analyze biological samples in situ. Thus, little or no sample preparation is required. Raman spectroscopy techniques can be readily performed in aqueous environments because water exhibits very little, but predictable, Raman scattering. It is particularly amenable to in vivo measurements as the powers and excitation wavelengths used are non-destructive to the tissue and have a relatively large penetration depth.

Raman chemical imaging (RCI) is a reagentless tissue imaging approach based on the scattering of laser light from tissue samples. The approach yields an image of a sample wherein each pixel of the image is the Raman spectrum of the sample at the corresponding location. The Raman spectrum carries information about the local chemical environment of the sample at each location. RCI has a spatial resolving power of approximately 250 nm and can potentially provide qualitative and quantitative image information based on molecular composition and morphology.

The vast majority of diseases, in particular cancer cases, are pathologically diagnosed using tissue from a biopsy specimen. Therefore it is desirable to devise systems and methodologies that use spectroscopic techniques to diagnose biological samples. It would be advantageous to devise systems and methods that would provide for the identification of regions of a sample that may be of interest. These regions may then be further investigated for a diagnosis of the sample.

### SUMMARY

The present disclosure provides for a system and method for assessing biological samples. More specifically, the invention of the present disclosure provides for the use of fluorescence and Raman spectroscopic and imaging techniques to diagnose biological samples. The invention disclosed herein provides for the use of fluorescence techniques to locate an area of interest of a biological sample. This area of interest may then be further evaluated by applying Raman techniques. Such evaluation holds potential for determining a diagnosis which may include, but is not limited to, at least one of: a metabolic state, a clinical outcome, a disease progression, a disease state, and combinations thereof.

### BRIEF DESCRIPTION OF THE DRAWINGS

The accompanying drawings, which are included to provide further understanding of the disclosure and are incorporated in an constitute a part of this specification illustrate embodiments of the disclosure, and together with the description, serve to explain the principles of the disclosure.

FIG. 1 is a schematic representation of an exemplary system of the present disclosure.

FIGS. 2A and 2B are schematic representations of an exemplary spectroscopy module of the present disclosure.

FIG. 3 is a schematic representation of an exemplary system of the present disclosure.

FIG. 4 is illustrative of a method of the present disclosure.

FIG. 5 is illustrative of the detection capabilities of the present disclosure.

### DETAILED DESCRIPTION

Reference will now be made in detail to the preferred embodiments of the present disclosure, examples of which are illustrated in the accompanying drawings. Wherever possible, the same reference numbers will be used throughout the specification to refer to same or like parts.

The patent or application file contains at least one drawing executed in color. Copies of this patent or patent application publication with color drawing(s) will be provided by the Office upon request and payment of the necessary fee.

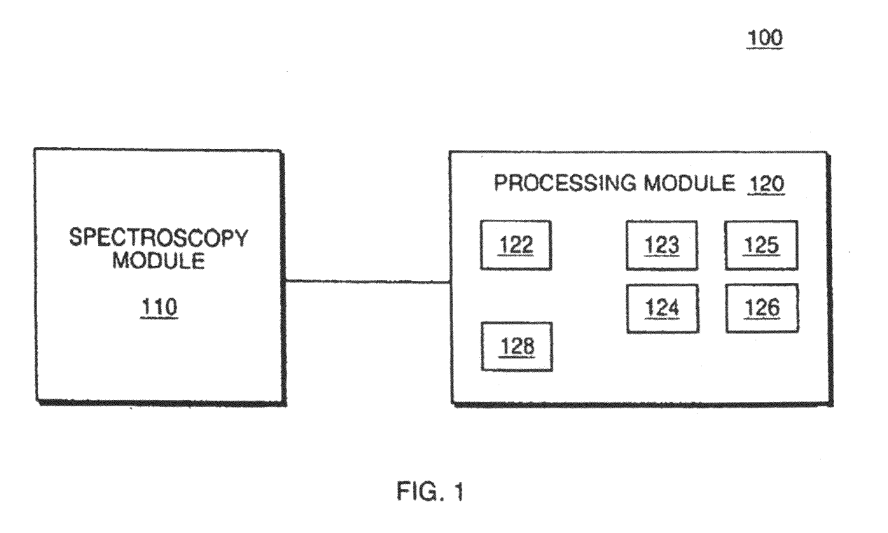

FIG. 1 illustrates an exemplary system 100 according to one embodiment of the present disclosure. System 100 includes a spectroscopy module 110 in communication with a processing module 120. Processing module 120 may include a processor 122, databases 123, 124, 125 and 126, and machine readable program code 128. The machine readable program code 128 may contain executable program instructions, and the processor 122 may be configured to execute the machine readable program code 128 so as to perform the methods of the present disclosure. In one embodiment, the program code 128 may contain the ChemImage Xpert.™. software marketed by ChemImage Corporation of Pittsburgh, Pa. The Xpert.™. software may be used to process spectroscopic data and information received from the spectroscopy module 110 to obtain various spectral plots and images, and to also carry out various multivariate image analysis methods discussed later herein below.

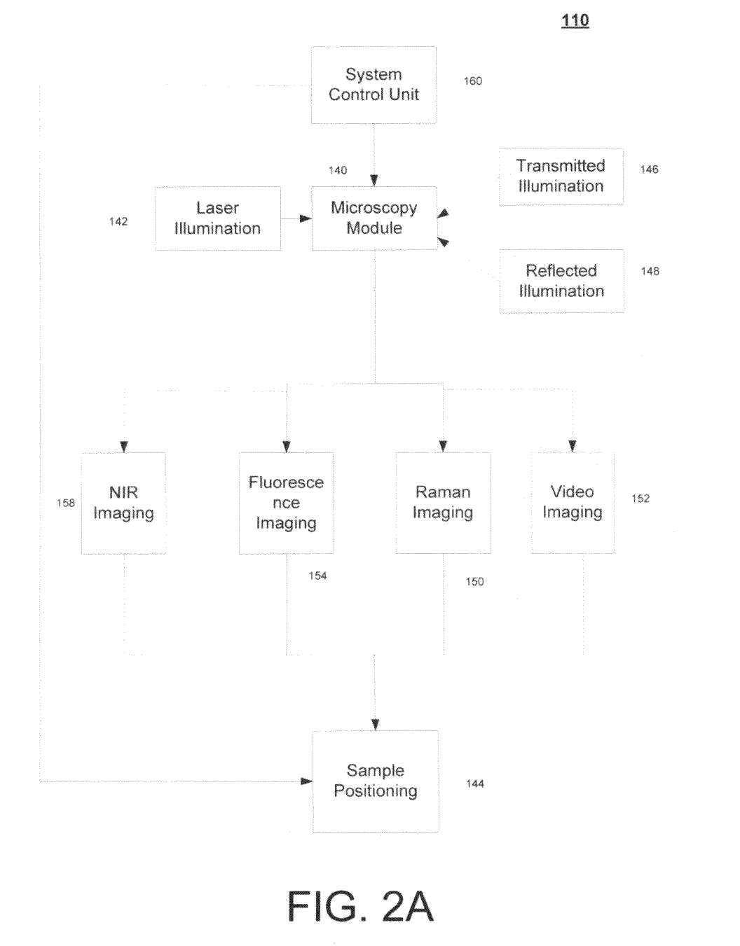

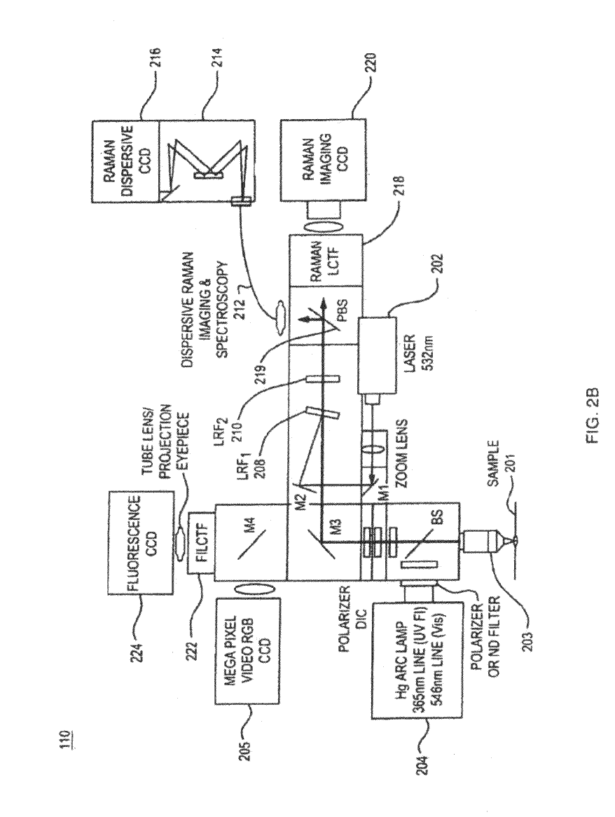

FIG. 2A illustrates an exemplary schematic layout of the spectroscopy module 110 shown in FIG. 1. The layout in FIG. 2A may relate to the Falcon II.™. Raman chemical imaging system marketed by ChemImage Corporation of Pittsburgh, Pa. In one embodiment, the spectroscopy module 110 may include a microscope module 140 containing optics for microscope applications. An illumination source 142 (e.g., a laser illumination source) may provide illuminating photons to a sample (not shown) handled by a sample positioning unit 144 via the microscope module 140. In one embodiment, photons transmitted, reflected, emitted, or scattered from the illuminated sample (not shown) may pass through the microscope module (as illustrated by exemplary blocks 146, 148 in FIG. 2A) before being directed to one or more of spectroscopy or imaging optics in the spectroscopy module 110. In the embodiment of FIG. 2A, Raman imaging 150 and fluorescence imaging 154 are illustrated as standard. In another embodiment, the modes of NIR imaging 158 and video imaging 152 may also be implemented. The spectroscopy module 110 may also include a control unit 160 to control operational aspects (e.g., focusing, sample placement, laser beam transmission, etc.) of various system components including, for example, the microscope module 140 and the sample positioning unit 144 as illustrated in FIG. 2A. In one embodiment, operation of various components (including the control unit 160) in the spectroscopy module 110 may be fully automated or partially automated, under user control.

It is noted here that in the discussion herein the terms “illumination,” “illuminating,” “irradiation,” and “excitation” are used interchangeably as can be evident from the context. For example, the terms “illumination source,” “light source,” and “excitation source” are used interchangeably. Similarly, the terms “illuminating photons” and “excitation photons” are also used interchangeably. Furthermore, although the discussion hereinbelow focuses more on Raman spectroscopy and Raman molecular imaging, various methodologies discussed herein may be adapted to be used in conjunction with other types of spectroscopy applications as can be evident to one skilled in the art based on the discussion provided herein.

FIG. 2B illustrates exemplary details of the spectroscopy module 110 in FIG. 2A according to one embodiment of the present disclosure. Spectroscopy module 110 may operate in several experimental modes of operation including bright field reflectance and transmission imaging, polarized light imaging, differential interference contrast (DIC) imaging, UV induced autofluorescence imaging, NIR imaging, wide field illumination whole field Raman spectroscopy, wide field spectral fluorescence imaging, and wide field spectral Raman imaging. Module 110 may include collection optics 203, light sources 202 and 204, and a plurality of spectral information processing devices including, for example: a tunable fluorescence filter 222, a tunable Raman filter 218, a dispersive spectrometer 214, a plurality of detectors including a fluorescence detector 224, and Raman detectors 216 and 220, a fiber array spectral translator (“FAST”) device 212, filters 208 and 210, and a polarized beam splitter (PBS) 219.

A FAST device may comprise a two-dimensional array of optical fibers drawn into a one-dimensional fiber stack so as to effectively convert a two-dimensional field of view into a curvilinear field of view, and wherein said two-dimensional array of optical fibers is configured to receive said photons and transfer said photons out of said fiber array spectral translator device and to at least one of: a spectrometer, a filter, a detector, and combinations thereof.

The FAST device can provide faster real-time analysis for rapid detection, classification, identification, and visualization of, for example, explosive materials, hazardous agents, biological warfare agents, chemical warfare agents, and pathogenic microorganisms, as well as non-threatening objects, elements, and compounds. FAST technology can acquire a few to thousands of full spectral range, spatially resolved spectra simultaneously, This may be done by focusing a spectroscopic image onto a two-dimensional array of optical fibers that are drawn into a one-dimensional distal array with, for example, serpentine ordering. The one-dimensional fiber stack may be coupled to an imaging spectrometer, a detector, a filter, and combinations thereof. Software may be used to extract the spectral/spatial information that is embedded in a single CCD image frame.

One of the fundamental advantages of this method over other spectroscopic methods is speed of analysis. A complete spectroscopic imaging data set can be acquired in the amount of time it takes to generate a single spectrum from a given material. FAST can be implemented with multiple detectors. Color-coded FAST spectroscopic images can be superimposed on other high-spatial resolution gray-scale images to provide significant insight into the morphology and chemistry of the sample.

The FAST system allows for massively parallel acquisition of full-spectral images. A FAST fiber bundle may feed optical information from is two-dimensional non-linear imaging end (which can be in any non-linear configuration, e.g., circular, square, rectangular, etc.) to its one-dimensional linear distal end. The distal end feeds the optical information into associated detector rows. The detector may be a CCD detector having a fixed number of rows with each row having a predetermined number of pixels. For example, in a 1024-width square detector, there will be 1024 pixels (related to, for example, 1024 spectral wavelengths) per each of the 1024 rows.

The construction of the FAST array requires knowledge of the position of each fiber at both the imaging end and the distal end of the array. Each fiber collects light from a fixed position in the two-dimensional array (imaging end) and transmits this light onto a fixed position on the detector (through that fiber's distal end).

Each fiber may span more than one detector row, allowing higher resolution than one pixel per fiber in the reconstructed image. In fact, this super-resolution, combined with interpolation between fiber pixels (i.e., pixels in the detector associated with the respective fiber), achieves much higher spatial resolution than is otherwise possible. Thus, spatial calibration may involve not only the knowledge of fiber geometry (i.e., fiber correspondence) at the imaging end and the distal end, but also the knowledge of which detector rows are associated with a given fiber.

In one embodiment, a system of the present disclosure may comprise FAST technology available from ChemImage Corporation, Pittsburgh, Pa. This technology is more fully described in the following U.S. Patents, hereby incorporated by reference in their entireties: U.S. Pat. No. 7,764,371, filed on Feb. 15, 2007, entitled “System And Method For Super Resolution Of A Sample In A Fiber Array Spectral Translator System”; U.S. Pat. No. 7,440,096, filed on Mar. 3, 2006, entitled “Method And Apparatus For Compact Spectrometer For Fiber Array Spectral Translator”; U.S. Pat. No. 7,474,395, filed on Feb. 13, 2007, entitled “System And Method For Image Reconstruction In A Fiber Array Spectral Translator System”; and U.S. Pat. No. 7,480,033, filed on Feb. 9, 2006, entitled “System And Method For The Deposition, Detection And Identification Of Threat Agents Using A Fiber Array Spectral Translator”.

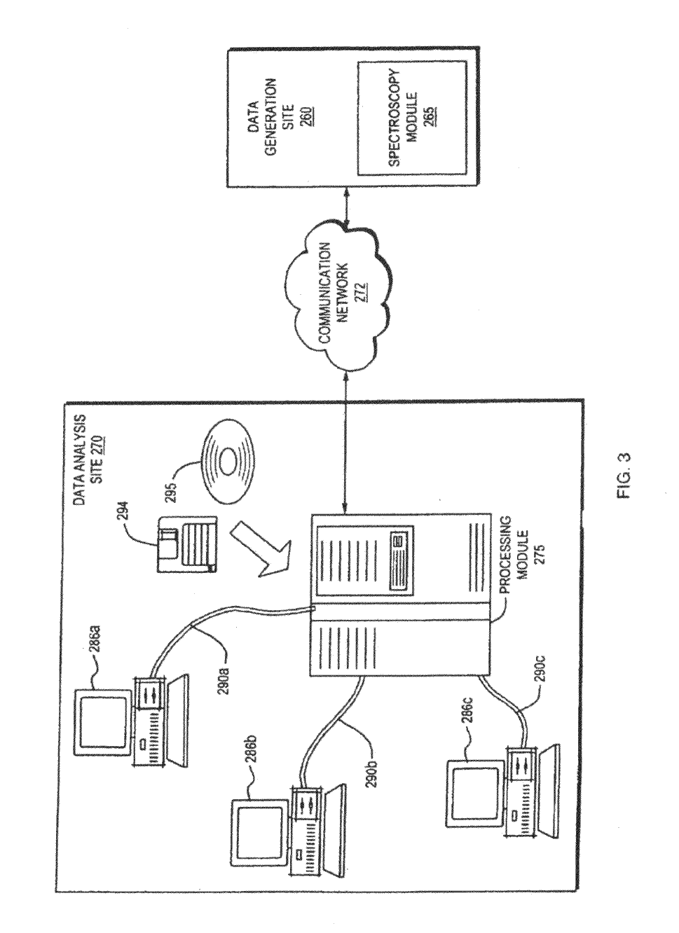

In one embodiment, the processor 122 (FIG. 1) may be operatively coupled to light sources 202 and 204, and the plurality of spectral information processing devices 214, 218 and 222. In another embodiment, the processor 122 (FIG. 1), when suitably programmed, can configure various functional parts of the spectroscopy module in FIG. 1 and may also control their operation at run time. The processor, when suitably programmed, may also facilitate various remote data transfer and analysis operations discussed in conjunction with FIG. 3. Module 110 may optionally include a video camera 205 for video imaging applications. Although not shown in FIG. 2B, spectroscopy module 110 may include many additional optical and electrical components to carry out various spectroscopy and imaging applications supported thereby.

A sample 201 may be placed at a focusing location (e.g., by using the sample positioning unit 144 in FIG. 2A) to receive illuminating photons and to also provide reflected, emitted, scattered, or transmitted photons from the sample 201 to the collection optics 203. Sample 201 may include a variety of biological samples. In one embodiment, the sample 201 includes at least one cell or a tissue containing a plurality of cells. The sample may contain normal (non-diseased or benign) cells, diseased cells (e.g., cancerous tissues with or without a progressive cancer state or malignant cells with or without a progressive cancer state) or a combination of normal and diseased cells. In one embodiment, the cell/tissue is a mammalian cell/tissue. Some examples of biological samples may include prostate cells, kidney cells, lung cells, colon cells, bone marrow cells, brain cells, red blood cells, and cardiac muscle cells. In one embodiment, the biological sample may include prostate cells. In one such embodiment, the biological sample may include Gleason 6 prostate cells: In another such embodiment, the biological sample may include Gleason 7 prostate cells. In another embodiment the biological sample may include a renal sample. In one such embodiment, the biological sample may include renal oncocytoma cells. In another such embodiment, the biological sample may include chromophobe renal carcinoma. In another embodiment, the sample 201 may include cells of plants, non-mammalian animals, fungi, protists, and monera. In yet another embodiment, the sample 201 may include a test sample (e.g., a biological sample under test to determine its metabolic state or its disease status or to determine whether it is cancerous state would progress to the next level). The “test sample,” “target sample” or unknown sample are used interchangeably herein to refer to a biological sample or renal sample under investigation, wherein such interchange use may be without reference to such biological sample's metabolic state or disease status.

A progressive cancer state is a cancer that will go on to become aggressive and acquire subsequent treatment by more aggressive means in order for the patient to survive. An example of progressive cancer is a Gleason score 7 cancer found in a prostate which has been surgically removed, where the patient, subsequent to the removal of the prostate, develops metastatic cancer. In this example the cancer progressed even after the removal of the source organ. Progressive cancers can be detected and identified in other organs and different types of cancer.

A non-progressive cancer is a cancer that does not progress to more advanced disease, requiring aggressive treatment. Many prostate cancers are non-progressive by this definition because though they are cancer by standard histopathological definition, they do not impact the life of the patient in a way that requires significant treatment. In many cases such cancers are observed and treated only if they show evidence of becoming progressive. Again, this is not a state particular to prostate cancer. Cancer cells are present in tissues of many health people. Because these do not ever transition to a state where they become progressive in terms of growth, danger to the patient, or inconvenience to the patient they would be considered non-progressive as the term is used herein.

The designation of progressive vs. non progressive can also be extended to other disease or metabolic states. As an example, diabetes can be clinically described as “stable”, “well managed” by a clinician and would fall into the non-progressive class. In contrast diabetes can be progressing through the common course of the disease with all of the effects on kidneys, skin, nerves, heart and other organs which are part of the disease. As a second example multiple sclerosis is a disease which exists in many people is a stable, non-progressive state. In some people the disease rapidly progresses through historically observed pattern of physical characteristics with clinical manifestations.

The cells can be isolated cells, such as individual blood cells or cells of a solid tissue that have been separated from other cells of the tissue (e.g., by degradation of the intracellular matrix). The cells can also be cells present in a mass, such as a bacterial colon/.y grown on a semi-solid medium or an intact or physically disrupted tissue. By way of example, blood drawn from a human can be smeared on the surface of a suitable Raman scattering substrate (e.g., an aluminum-coated glass slide) and individual cells in the sample can be separately imaged by light microscopy and Raman scattering analysis using the spectroscopy module 110 of FIG. 2B. Similarly a slice of a solid tissue (e.g., a piece of fresh tissue or a paraffin-embedded thin section of a tissue) can be imaged on a suitable surface.

The cells can be cells obtained from a subject (e.g., cells obtained from a human blood or urine sample, semen sample, tissue biopsy, or surgical procedure). Cells can also be studied where they naturally occur, such as cells in an accessible location (e.g., a location on or within a human body), cells in a remote location using a suitable probe, or by revealing cells (e.g., surgically) that are not normally accessible.

Referring again to FIG. 2B, light source 202 may be used to irradiate the sample 201 with substantially monochromatic light. Light source 202 can include any conventional photon source, including, for example, a laser, an LED (light emitting diode), or other IR (infrared) or near IR (NIR) devices. The substantially monochromatic radiation reaching sample 201 illuminates the sample 201, and may produce photons scattered from different locations on or within the illuminated sample 201. A portion of the Raman scattered photons from the sample 201 may be collected by the collection optics 203 and directed to dispersive spectrometer 214 or Raman tunable filter 218 for further processing discussed later herein below. In one embodiment, light source 202 includes a laser light source producing light at 532.1 nm. The laser excitation signal is focused on the sample 201 through combined operation of reflecting mirrors M1, M2, M3, the filter 208, and the collection optics 203 as illustrated by an exemplary optical path in the embodiment of FIG. 2B. The filter 208 may be tilted at a specific angle from the vertical (e.g., at 6.5.sup.0) to reflect laser illumination onto the mirror M3, but not to reflect Raman-scattered photons received from the sample 201. The other filter 210 may not be tilted (i.e., it remains at 0.sup.0 from the vertical). Filters 208 and 210 may function as laser line rejection filters to reject light at the wavelength of laser light source 202.

In the spectroscopy module 110 in the embodiment of FIG. 2B, the second light source 204 may be used to irradiate the sample 201 with ultraviolet light or visible light. In one embodiment, the light source 204 includes a mercury arc (Hg arc) lamp that produces ultraviolet radiation (UV) having wavelength at 365 nm for fluorescence spectroscopy applications. In yet another embodiment, the light source 204 may produce visible light at 546 nm for visible light imaging applications. A polarizer or neutral density (ND) filter with or without a beam splitter (BS) may he provided in front of the light source 204 to obtain desired illumination light intensity and polarization.

In the embodiment of FIG. 2B, the dispersive spectrometer 214 and the Raman tunable filter 218 function to produce Raman data sets of sample 201. A Raman data set corresponds to one or more of the following: a plurality of Raman spectra of the sample; and a plurality of spatially accurate wavelength resolved Raman images of the sample. In one embodiment, the plurality of Raman spectra is generated by dispersive spectral measurements of individual cells. In this embodiment, the illumination of the individual cell may cover the entire area of the cell so the dispersive Raman spectrum is an integrated measure of spectral response from all the locations within the cell.

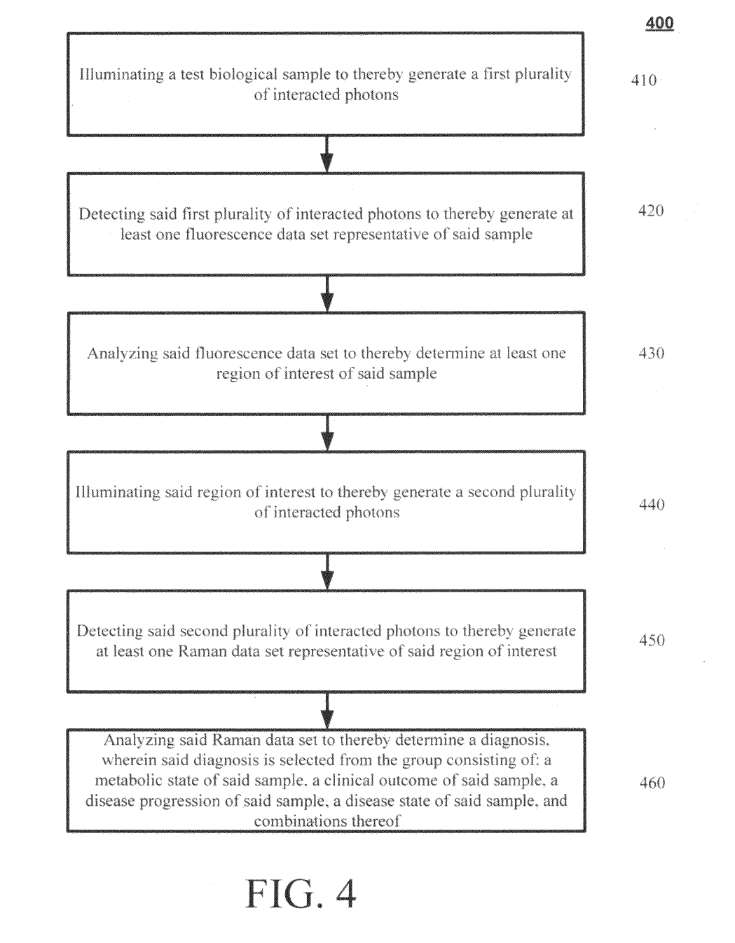

In another embodiment, the Raman data set corresponds to a three dimensional block of Raman data (e.g., a spectral hypercube or a Raman image) having spatial dimensional data represented in the x and y dimensions and wavelength data represented in the z dimension as exemplified in FIGS. 4A-4C. Each Raman image has a plurality of pixels where each has a corresponding x and y position in the Raman image. The Raman image may have one or more regions of interest. The regions of interest may be identified by the size and shape of one or more pixels and is selected where the pixels are located within the regions of interest. A single Raman spectrum is then extracted from each pixel located in the region of interest, leading to a plurality of Raman spectra for each of the regions of interest. The extracted plurality of Raman spectra are then designated as the Raman data set. In this embodiment, the plurality of Raman spectra and the plurality of spatially accurate wavelength resolved Raman images are generated, as components of the hypercube, by a combination of the Raman tunable filter 218 and Raman imaging detector 220 or by a combination of the FAST device 212, the dispersive spectrometer 214, and the Raman detector 216.

In yet another embodiment, a Raman dataset is generated using a Raman image to identify one or more regions of interest of the sample 201. In one such embodiment, the one or more regions of interest contain at least one of the following: an epithelium area, a stroma area, epithelial-stromal junction (ESJ) area and/or nuclei area. A plurality of Raman spectra may be obtained from the one or more of regions of interest of the sample 201. In standard operation the Raman spectrum generated by selecting a region of interest in a Raman image is the average spectrum of all the spectra at each pixel within the region of interest. The standard deviation between of all the spectra in the region of interest may be displayed along with the average Raman spectrum of the region of interest. Alternatively, all of the spectra associated with pixels within a region can be considered as a plurality of spectra, without the step of reducing them to a mean and standard deviation.

With further reference to FIG. 2B, the fluorescence tunable filter 222 may function to produce fluorescence data sets of the photons emitted from the sample 201 under suitable illumination (e.g., UV illumination). In one embodiment, the fluorescence data set includes a plurality of fluorescence spectra of sample 201 and/or a plurality of spatially accurate wavelength resolved fluorescence images of sample 201. A fluorescence spectrum of sample 210 may contain a fluorescence emission signature of the sample 201. In one embodiment, the emission signature may be indicative of a fluorescent probe (e.g., fluorescein isothiocyanate) within the sample 201. The fluorescence data sets may be detected by fluorescence CCD detector 224. A portion of the fluorescence emitted photons or visible light reflected photons from the sample 201 may be directed to the video imaging camera 205 via a mirror M4 and appropriate optical signal focusing mechanism.

In one embodiment, a microscope objective (including the collection optics 203) may be automatically or manually zoomed in or out to obtain proper focusing of the sample.

The entrance slit (not shown) of the spectrometer 214 may be optically coupled to the output end of the fiber array spectral translator device 212 to disperse the Raman scattered photons received from the FAST device 212 and to generate a plurality of spatially resolved Raman spectra from the wavelength-dispersed photons. The FAST device 212 may receive Raman scattered photons from the beam splitter 219, which may split and appropriately polarize the Raman scattered photons received from the sample 201 and transmit corresponding portions to the input end of the FAST device 212 and the input end of the Raman tunable filter 218.

Referring again to FIG. 2B, the tunable fluorescence filter 222 and the tunable Raman filter 218 may be used to individually tune specific photon wavelengths of interest and to thereby generate a plurality of spatially accurate wavelength resolved spectroscopic fluorescence images and Raman images, respectively, in conjunction with corresponding detectors 224 and 220. In one embodiment, each of the fluorescence filter 222 and the Raman filter 218 includes a two-dimensional tunable filter, such as, for example, an electro-optical tunable filter, a liquid crystal tunable filter (LCTF), or an acousto-optical tunable filter (AOTF). A tunable filter may be a band-pass or narrow band filter that can sequentially pass or “tune” fluorescence emitted photons or Raman scattered photons into a plurality of predetermined wavelength bands. The plurality of predetermined wavelength bands may include specific wavelengths or ranges of wavelengths. In one embodiment, the predetermined wavelength bands may include wavelengths characteristic of the sample undergoing analysis. The wavelengths that can be passed through the fluorescence filter 222 and Raman filter 218 may range from 200 nm (ultraviolet) to 2000 nm (i.e., the far infrared). The choice of a tunable filter depends on the desired optical region and/or the nature of the sample being analyzed. Additional examples of a two-dimensional tunable filter may include a Fabry Perot angle tuned filter, a Lyot filter, an Evans split element liquid crystal tunable filter, a Solc liquid crystal tunable filter, a spectral diversity filter, a photonic crystal filter, a fixed wavelength Fabry Perot tunable filter, an air-tuned Fabry Perot tunable filter, a mechanically-tuned Fabry Perot tunable filter, and a liquid crystal Fabry Perot tunable filter. As noted before, the tunable filters 218, 222 may be selected to operate in one or more of the following spectral ranges: the ultraviolet (UV), visible, and near infrared. In one such embodiment, the tunable filters 218, 222 may be selected to operate in spectra ranges of 900-1155 cm-.sup.1 and 15-30-1850 cm-.sup.1 Raman shift values.

In one embodiment, a multi-conjugate filter (MCF) may be used instead of a simple LCTF (e.g., the LCTF 218 or 222) to provide more precise wavelength tuning of photons received from the sample 201. Some exemplary multi-conjugate filters are discussed, for example, in U.S. Pat. No. 6,992,809, titled “Multi-Conjugate Liquid Crystal Tunable Filter;” and in the United States Published Patent Application Number US2007/0070260A1, titled “Liquid Crystal Filter with Tunable Rejection Band,” the disclosures of both of these publications are incorporated herein by reference in their entireties.

In the embodiment of FIG. 2B, the fluorescence spectral data sets (output from the tunable filter 222) may be detected by the detector 224, and the Raman spectral data sets (output from the spectrometer 214 and the tunable filter 218) may be detected by detectors 216 and 220. The detectors 216, 220, and 224 may detect received photons in a spatially accurate manner. Detectors 216, 220 and 224 may include an optical signal (or photon) collection device such as, for example, an image focal plane array (FPA) detector, a charge coupled device (CCD) detector, or a CMOS (Complementary Metal Oxide Semiconductor) array sensor. Detectors 216, 220 and 224 may measure the intensity of scattered, transmitted or reflected light incident upon their sensing surfaces (not shown) at multiple discrete locations or pixels, and transfer the spectral information received to the processor module 120 for storage and analysis. The optical region employed to characterize the sample of interest governs the choice of two-dimensional array detector. For example, a two-dimensional array of silicon charge-coupled device (CCD) detection elements can be employed with visible wavelength emitted or reflected photons, or with Raman scatter photons, while gallium arsenide (GaAs) and gallium indium arsenide (GaInAs) FPA detectors can be employed for image analyses at near infrared wavelengths. The choice of such devices may also depend on the type of sample being analyzed.

In one embodiment, a display unit (not shown) may be provided to display spectral data collected by various detectors 216, 220, 224 in a predefined or user-selected format. The display unit may be a computer display screen, a display monitor, an LCD (liquid crystal display) screen, or any other type of electronic display device.

Referring again to FIG. 1, the databases 123-126 may store various reference spectral data sets including, for example, a reference Raman data set, a reference fluorescence data set, a reference NIR data set, etc. The reference data sets may be collected from different samples and may be used to detect or identify the sample 201 from comparison of its spectral data set with the reference data sets. In one embodiment, during operation, the Raman data sets and fluorescence data sets of the sample 201 also may be stored in one or more of the databases (e.g., database 123) of the processing module 120.

For example, in one embodiment, database 123 may be used to store a plurality of reference Raman data sets from reference cells having a known metabolic state or a known disease state. In one such embodiment, the reference Raman data sets may correspond to a plurality of reference Raman spectra. In another such embodiment, the reference Raman data sets may correspond to a plurality of reference spatially accurate wavelength resolved Raman images.

In another embodiment, the database 124 may be used to store a first plurality of reference Raman data sets from reference normal (non-diseased) cells. In one embodiment, the first reference set of Raman data sets may include a plurality of first reference Raman spectra. In another embodiment, a first reference Raman spectrum may correspond to a dispersive Raman spectrum. In a further embodiment, the first reference set of Raman data sets may include a plurality of first reference spatially accurate wavelength resolved Raman images obtained from corresponding reference normal cells.

In yet another embodiment, the database 125 may store a second plurality of reference Raman data sets from different types of reference diseased cells. In one such embodiment, the reference diseased cells correspond to chromophobe renal carcinoma cells. In one embodiment, the second reference set of Raman data sets includes a plurality of second reference Raman spectra. In one embodiment, the second reference Raman spectrum may correspond to a dispersive Raman spectrum. In another embodiment, the second reference set of Raman data sets may include a plurality of second reference spatially accurate wavelength resolved Raman images obtained from corresponding reference diseased cells.

Similarly, database 126 may store a plurality of reference fluorescence spectra and/or a plurality of reference spatially accurate wavelength resolved fluorescence spectroscopic images obtained from reference biological samples (e.g., cancerous human tissues). One or more of the reference biological samples may include fluorescence probe molecules (e.g., fluorescein isothiocyanate). In one embodiment, a single database may be used to store all types of spectra.

The reference Raman data sets may be associated with a reference Raman image and/or a corresponding reference non-Raman image. In one such embodiment, the reference non-Raman image may include at least one of: a brightfield image; a polarized light image; and a UV-induced autofluorescence image.

FIG. 3 depicts an exemplary setup to remotely perform spectroscopic analysis of test samples according to one embodiment of the present disclosure. Spectroscopic data from a test sample or a test sample may be collected at a data generation site 260 using a spectroscopy module 265. In one embodiment, the spectroscopy module may be functionally similar to the spectroscopy module 110 discussed hereinbefore with reference to FIGS. 2A-2B. The spectroscopic data collected at the data generation site 260 may be transferred to a data analysis site 270 via a communication network 272. In one embodiment, the communication network 272 may be any data communication network such as an Ethernet LAN (local area network) connecting all the data processing and computing units within a facility, e.g., a university research laboratory, or a corporate research center. In that case, the data generation site 260 and the data analysis site 270 may be physically located within the same facility, e.g., a university research laboratory or a corporate research center. In alternative embodiments, the communication network 272 may include, independently or in combination, any of the present or future wireline or wireless data communication networks such as, for example, the Internet, the PSTN (public switched telephone network), a cellular telephone network, a WAN (wide area network), a satellite-based communication link, a MAN (metropolitan area network), etc. In this case, the data generation site 260 and the data analysis site 270 may be physically located in different facilities. In some embodiments, the data generation site 260 and the data analysis site 270 that are linked by the communication network 272 may be owned or operated by different entities.

The data analysis site 270 may include a processing module 275 to process the spectroscopic data received from the data generation site 260. In one embodiment, the processing module 275 may be similar to the processing module 120 and may also include a number of different databases (not shown) storing different reference spectroscopic data sets (e.g., a first plurality of reference Raman data sets for non-progressive cancer tissues, a second plurality of reference Raman data sets for progressive cancer tissues, a third plurality of reference Raman data sets for normal or non-diseased tissues; a fourth plurality of reference data set for renal oncocytomas samples and chromophobe renal cell carcinoma samples, etc.). The processing module 275 may include a processor (similar to the processor 122 of the processing module 120 in FIG. 1) that is configured to execute program code or software to perform various spectral data processing tasks according to the teachings of the present disclosure. The machine-readable program code containing executable program instructions may be initially stored on a portable data storage medium, e.g., a floppy diskette 294, a compact disc or a DVD 295, a data cartridge tape (not shown), or any other suitable digital data storage medium. The processing module 275 may include appropriate disk drives to receive the portable data storage medium and may be configured to read the program code stored thereon, thereby facilitating execution of the program code by its processor. The program code, upon execution by the processor of the processing module 275, may cause the processor to perform a variety of data processing and display tasks including, for example, initiate transfer of spectral data set from the data generation site 260 to the data analysis site 270 via the communication network 272, compare the received spectral data set to various reference data sets stored in the databases of the processing module 275, classify or identify the test sample based on the comparison (e.g., whether the test sample has a progressive cancer or non-progressive cancer state or whether the test sample has renal oncocytomas disease or chromophobe renal cell carcinoma disease), transfer the classification or identification results to the data generation site 260 via the communication network 272, etc.

In one embodiment, the data analysis site 270 may include one or more computer terminals 286A-286C communicatively connected to the processing module 275 via corresponding data communication links 290A-290C, which can be serial, parallel, or wireless communication links, or a suitable combination thereof. Thus, users may utilize functionalities of the processing module 275 via their computer terminals 286A-286C, which may also be used to display spectroscopic data received from the data generation site 260 and the results of the spectroscopic data processing by the processing module 275, among other applications. It is evident that in a practical application, there may be many more computer terminals 286 than just three terminals shown in FIG. 3.

The computer terminals 286A-286C may be, e.g., a personal computer (PC), a graphics workstation, a multiprocessor computer system, a distributed network of computers, or a computer chip embedded as part of a machine or mechanism. Similarly, the data generation site 260 may include one or more of such computers (not shown) for viewing the results of the spectroscopic analysis received from the data analysis site 270. Each computer terminal, whether at the data generation site 260 or at the data analysis site 270, may include requisite data storage capability in the form of one or more volatile and non-volatile memory modules. The memory modules may include RAM (random access memory), ROM (read only memory) and HDD (hard disk drive) storage.

It is noted that the arrangement depicted in FIG. 3 may be used to provide a commercial, network-based spectroscopic data processing service that may perform customer-requested processing of spectroscopic data in real time or near real time. For example, the processing module 275 at the data analysis site 270 may be configured to identify a test sample from the spectroscopic data remotely submitted to it over the communication network 272 (e.g., the Internet) from the spectroscopy module 265 automatically or through an operator at the data generation site 260. The client site (data generation site) 260 may be, for example, a government laboratory or a medical facility or pathological laboratory. The results of spectroscopic data analysis may be transmitted back to the client site 260 for review and further analysis. In one embodiment, the whole data submission, analysis, and reporting process can be automated.

It is further noted that the owner or operator of the data analysis site 270 may commercially offer a network-based spectroscopic data content analysis service, as illustrated by the arrangement in FIG. 3, to various individuals, corporations, governmental entities, laboratories, or other facilities on a fixed-fee basis, on a per-operation basis or on any other payment plan mutually convenient to the service provider and the service recipient.

Processing module 120 may also include a test Raman database associated with a test biological sample having an unknown metabolic state. In one such embodiment, the test Raman data set may correspond to a plurality of Raman spectra of the test biological sample. In another such embodiment, the test Raman data set may correspond to a plurality of spatially accurate wavelength resolved Raman images of the test biological sample. In another embodiment, each of the test Raman data sets may be associated with least one of the following: a corresponding test Raman image; and a corresponding test non-Raman image. In one such embodiment, the test non-Raman image may include at least one of the following: a brightfield image; a polarized light image; and a UV-induced autofluorescence image.

In one such embodiment, processing module 120 may also include a test Raman database associated with a test biological sample having an unknown diagnosis. In one such embodiment, the test Raman data set may correspond to a plurality of Raman spectra of the test biological sample. In another such embodiment, the test Raman data set may correspond to a plurality of spatially accurate wavelength resolved Raman images of the test biological sample. In another embodiment, each of the test Raman data sets may be associated with least one of the following: a corresponding test Raman image; and a corresponding test non-Raman image. In one such embodiment, the test non-Raman image may include at least one of the following: a brightfield image; a polarized light image; and a UV-induced autofluorescence image.

In one embodiment, the test Raman spectra are generated using a test Raman image to identify one or more regions of interest of the test biological sample. In one such embodiment, the one or more regions of interest contain at least one of the following: an epithelium area, a stroma area, epithelial-stromal junction (ESJ) area, and/or nuclei area. A plurality of test Raman spectra may be obtained from the one or more of regions of interest of the test biological sample.

A diagnosis of a test sample as diseased or non-diseased or a prediction of the metabolic state of a test sample may be made by comparing a test Raman data set to reference Raman data sets using a chemometric technique. In one embodiment, the chemometric technique may be spectral unmixing. The application of spectral unmixing to determine the identity of components of a mixture is described in U.S. Pat. No. 7,072,770, entitled “Method for Identifying Components of a Mixture via Spectral Analysis, issued on Jul. 4, 2006, which is incorporated herein by reference in it entirety. Spectral unmixing as described in the above referenced patent can be applied as follows: Spectral unmixing requires a library of spectra which include possible components of the test sample. The library can in principle be in the form of a single spectrum for each component, a set of spectra for each component, a single Raman image for each component, a set of Raman images for each component, or any of the above as recorded after a dimension reduction procedure such as Principle Component Analysis. In the methods discussed herein, the library used as the basis for application of spectral unmixing is the reference Raman data sets.

With this as the library, a set of Raman measurements made on a sample of unknown state, described herein as a test Raman data set, is assessed using the methods of U.S. Pat. No. 7,072,770 to determine the most likely groups of components which are present in the sample. In this instance the components are actually disease states of interest and/or clinical outcome. The result is a set of disease state groups and/or clinical outcome groups with a ranking of which are most likely to be represented by the test data set.

Given a set of reference spectra, such as those described above, a piece or set of test data can be evaluated by a process called spectral mixture resolution. In this process, the test spectrum is approximated with a linear combination of reference spectra with a goal of minimizing the deviation of the approximation from the test spectrum. This process results in a set of relative weights for the reference spectra.

In one embodiment, the chemometric technique may be Principal Component Analysis. Using Principal Component Analysis results in a set of mathematical vectors defined based on established methods used in multivariate analysis. The vectors form an orthogonal basis, meaning that they are linearly independent vectors. The vectors are determined based on a set of input data by first choosing a vector which describes the most variance within the input data. This first “principal component” or PC is subtracted from each of the members of the input set. The input set after this subtraction is then evaluated in the same fashion (a vector describing the most variance in this set is determined and subtracted) to yield a second vector--the second principal component. The process is iterated until either a chosen number of linearly independent vectors (PCs) are determined, or a chosen amount of the variance within the input data is accounted for.

In one embodiment, the Principal Component Analysis may include a series of steps. A pre-determined vector space is selected that mathematically describes a plurality of reference Raman data sets. Each reference Raman data set may be associated with a known biological sample having an associated metabolic state. The test Raman data set, may be transformed into the pre-determined vector space, and then a distribution of transformed data may be analyzed in the pre-determined vector space to generate a diagnosis.

In another embodiment, the Principal Component Analysis may include a series of steps. A pre-determined vector space is selected that mathematically describes a first plurality of reference Raman data sets associated with a known biological sample having an associated diseased state and a second plurality of reference Raman data sets associated with a known biological sample having an associated non-diseased state. The test Raman data set may be transformed into the pre-determined vector space, and then a distribution of transformed data may be analyzed in the pre-determined vector space to generate a diagnosis.

In yet another embodiment, the Principal Component Analysis may include a series of steps. A pre-determined vector space is selected that mathematically describes a first plurality of reference Raman data sets associated with a known biological sample having an associated progressive state and a second plurality of reference Raman data sets associated with a known biological sample having an associated non-progressive state. The test Raman data set may be transformed into the pre-determined vector space, and then a distribution of transformed data may be analyzed in the pre-determined vector space to generate a diagnosis.

In still yet another embodiment, the Principal Component Analysis may include a series of steps. A pre-determined vector space may be selected that mathematically describes a first plurality of reference Raman data sets associated with a known diagnosis. The test Raman data set may be transformed into the pre-determined vector space, and then a distribution of transformed data may he analyzed in the pre-determined vector space.

The analysis of the distribution of the transformed data may be performed using a classification scheme. Some examples of the classification scheme may include: Mahalanobis distance, Adaptive subspace detector, Band target entropy method, Neural network, and support vector machine as an incomplete list of classification schemes known to those skilled in the art.

In one such embodiment, the classification scheme is Mahalanobis distance. The Mahalanobis distance is an established measure of the distance between two sets of points in a multidimensional space that takes into account both the distance between the centers of two groups, but also the spread around each centroid. A Mahalanobis distance model of the data is represented by plots of the distribution of the spectra in the principal component space. The Mahalanobis distance calculation is a general approach to calculating the distance between a single point and a group of points. It is useful because rather than taking the simple distance between the single point and the mean of the group of points, Mahalanobis distance takes into account the distribution of the points in space as part of the distance calculation. The Mahalanobis distance is calculated using the distances between the points in all dimensions of the principal component space.

In one such embodiment, once the test Raman data is transformed into the space defined by the predetermined PC vector space, the test data is analyzed relative to the pre-determined vector space. This may be performed by calculating a Mahalanobis distance between the test Raman data set transformed into the pre-determined vector space and the Raman data sets in the pre-determined vector space to generate a diagnosis.

The exemplary systems of FIGS. 1 and 2 may be used to perform methods to predict the clinical outcome of patients or diagnose a disease state of patients. Processor 122 is configured to execute program instructions to carry out these methods. In another embodiment of the present disclosure, the exemplary system of FIG. 3 may be used to carry out methods to predict the clinical outcome of patients. In this method, data obtained at a data generation site is transmitted to an analysis site to obtain a prediction of the metabolic state of a test biological sample. The prediction is then transmitted back to the data generation site. The transmission may be performed over a data communication network such as the Internet.

One embodiment of the present disclosure, illustrated by FIG. 4, provides for a method for diagnosing a biological sample. In such an embodiment, the method 400 may comprise illuminating a test biological sample, in step 410, to thereby generate a first plurality of interacted photons. In one embodiment, this test biological sample may comprise at least one of: a kidney sample, a prostate sample, a breast sample, a pancreatic sample, a brain sample, a skin sample, an intestinal sample, a colon sample, a liver sample, a cardiac sample, a lung sample, an esophageal sample, a bladder sample, a blood sample, a urethral sample, an ovarian sample, a uterine sample, a testicular sample, a bone sample, a stomach sample, a tracheal sample, a tongue sample, a diaphragm sample, a nerve sample, and combinations thereof.

In one embodiment, the first plurality of interacted photons may comprise photons selected from the group consisting of: photons absorbed by said sample, photons reflected by said sample, photons scattered by said sample, photons emitted by said sample, and combinations thereof. This first plurality of interacted photons may be generated by illuminating said sample using UV light.

In step 420 this first plurality of interacted photons may be detected to thereby generate at least one fluorescence data set representative of said sample. In one embodiment, this fluorescence data set may comprise at least one of: a fluorescence spectrum, a spatially accurate wavelength resolved fluorescence image, and combinations thereof. In one embodiment, this fluorescence data set may comprise a hyperspectral fluorescence image representative of said sample. In one embodiment, this first plurality of interacted photons may be detected using a detector selected from the group consisting of: a focal plane array detector, a CCD, an ICCD, a CMOS detector, and combinations thereof.

This fluorescence data set may be analyzed in step 430 to thereby determine at least one region of interest of said sample. In one embodiment, this analysis may comprise visually assessing said fluorescence data set to thereby identify said region of interest. This region of interest may be such that it exhibits characteristic fluorescence.

In another embodiment, the analyzing of step 430 may be accomplished by comparing said fluorescence data set to at least one reference data set. In such an embodiment, the method 400 may further comprise providing a reference database comprising a plurality of reference data sets, each reference data set being associated with at least one known diagnosis. In one embodiment, each reference data set in said reference database may be associated with at least one of: a known metabolic state, a known clinical outcome, a known disease progression, a known disease state, and combinations thereof.

In one embodiment, this comparison may be achieved by applying at least one chemometric technique. This chemometric technique may comprise at least one of: principle component analysis, partial least squares discriminate analysis, cosine correlation analysis, Euclidian distance analysis, k-means clustering, multivariate curve resolution, band t. entropy method, mahalanobis distance, adaptive subspace detector, spectral mixture resolution, and combinations thereof. In one embodiment, the chemometric technique may be spectral unmixing. The application of spectral unmixing to determine the identity of components of a mixture is described in U.S. Pat. No. 7,072,770, entitled “Method for Identifying Components of a Mixture via Spectral Analysis, issued on Jul. 4, 2006, which is incorporated herein by reference in its entirety.

This region of interest may be illuminated in step 440 to thereby generate a second plurality of interacted photons. In one embodiment, this second plurality of interacted photons may comprise photons selected from the group consisting of: photons absorbed by said region of interest, photons reflected by said region of interest, photons scattered by said region of interest, photons emitted by said region of interest, and combinations thereof. In one embodiment, this second plurality of interacted photons may be generated by illuminating said region of interest using substantially monochromatic light.

In step 450 this second plurality of interacted photons may be detected to thereby generate at least one Raman data set representative of said region of interest. In one embodiment, this second plurality of interacted photons may be detected using a detector selected from the group consisting of: a CCD, an ICCD, a CMOS detector, and combinations thereof. In one embodiment, this Raman data set may comprise at least one of: a Raman spectrum, a spatially accurate wavelength resolved Raman image, and combinations thereof. In one embodiment, this Raman data set may comprise at least one hyperspectral Raman image representative of said region of interest.

This Raman data set may be analyzed in step 460 to thereby determine a diagnosis of said sample. In one embodiment, this diagnosis may comprise at least one of: a metabolic state of said sample, a clinical outcome of said sample, a disease progression of said sample, a disease state of said sample, and combinations thereof. This Raman data set may be analyzed by comparison to at least one reference data set. In one embodiment, this comparison may accomplished by applying at least one chemometric technique.

In one embodiment, the method 400 may further comprise passing at least one of said first plurality of interacted photons and said second plurality of interacted photons through a tunable filter. In one embodiment, this tunable filter may comprise filter technology available from ChemImage Corporation, Pittsburgh, Pa. This technology is more fully described in the following U.S. Patents and Patent Applications: U.S. Pat. No. No. 6,992,809, filed on Jan. 31, 2006, entitled “Multi-Conjugate Liquid Crystal Tunable Filter,” U.S. Pat. No. 7,362,489, filed on Apr. 22, 2008, entitled “Multi-Conjugate Liquid Crystal Tunable Filter,” U.S. Pat. No. 13/066,428, filed on Apr. 14, 2011, entitled “Short wave infrared multi-conjugate liquid crystal tunable filter.” These patents and patent applications are hereby incorporated by reference in their entireties.

In one embodiment, this tunable filter may comprise a filter selected from the group consisting of: a liquid crystal tunable filter, a multi-conjugate tunable filter, an acousto-optical tunable filter, a Lyot liquid crystal tunable filter, an Evans split-element liquid crystal tunable filter, a Solc liquid crystal tunable filter, a ferroelectric liquid crystal tunable filter, a Fabry Perot liquid crystal tunable filter, and combinations thereof.

In one embodiment, the method 400 may further comprise generating at least one of said fluorescence data set and said Raman data set at a data generation site. At least one of the fluoresce data set and the Raman data set may then be transmitted over a data communication network to an analysis center. At least one of the fluorescence data set and the Raman data set may then be analyzed to thereby determine a diagnosis. This diagnosis may be selected from the group consisting of: a metabolic state of the sample, a clinical outcome of the sample, a disease progression of the sample, a disease state of the sample, and combinations thereof. This diagnosis may then be transferred to said data generation site via the data communication network.

In one embodiment, the present disclosure also provides for a storage medium containing machine readable program code, which when executed by a processor, causes said processor to perform the following: illuminate a test biological sample to thereby generate a first plurality of interacted photons; detect said first plurality of interacted photons to thereby generate at least one fluorescence data set representative of said sample; analyze said fluorescence data set to thereby determine a region of interest of said sample; illuminate said region of interest to thereby generate a second plurality of interacted photons; detect said second plurality of interacted photons to thereby generate at least one Raman data set representative of said region of interest; and analyze said Raman data set to thereby determine at least one of: a metabolic state of said sample, a clinical outcome of said sample, a disease progression of said sample, a disease state of said sample, and combinations thereof.

In one embodiment of the present disclosure, the method 400 may further comprise fusing said fluorescence data set and said Raman data set to thereby generate a fused data set. In one embodiment, this fusion may be accomplished using Bayesian fusion. In one embodiment, this fusion may be accomplished using fusion technology available from ChemImage Corporation, Pittsburgh, Pa. This technology is more fully described in the following patent and published U.S. Patent Applications: US 2007/0192035, filed on Jun. 9, 2006, entitled “Forensic Integrated Search Technology;” US 2009/0012723, filed on Aug. 22, 2008, entitled “Adaptive Method for Outlier Detection and Spectral Library Augmentation;” US 2008/0300826, filed on Jan. 22, 2008, entitled “Forensic Integrated Search Technology With Instrument Weight Factor Determination.” These patents and patent applications are hereby incorporated by reference in their entireties.

In one embodiment, the method 400 may be automated using software. In one embodiment, the invention of the present disclosure may utilize machine readable program code which may contain executable program instructions. A processor may be configured to execute the machine readable program code so as to perform the methods of the present disclosure. In one embodiment, the program code may contain the ChemImage Xpert® software marketed by ChemImage Corporation of Pittsburgh, Pa. The ChemImage Xpert® software may be used to process image and/or spectroscopic data and information received from a system of the present disclosure to obtain various spectral plots and images, and to also carry out various multivariate image analysis methods discussed herein.

In one embodiment, said machine readable program code, when executed by a processor, may further cause said processor to pass at least one of said first plurality of interacted photons and said second plurality of interacted photons through a tunable filter. In one embodiment, said machine readable program code, further causes said processor to: compare at least one of said fluorescence data set and said Raman data set to at least one reference data set.

The present disclosure also contemplates a system for assessing a test biological sample to thereby determine a diagnosis. In one embodiment, this system may comprise a first illumination source configured so as to illuminate a test biological sample to thereby generate a first plurality of interacted photons. In one embodiment, this first illumination source may be configured so as to illuminate said test biological sample with UV light. A first detector may be configured so as to detect said first plurality of interacted photons and generate at least one fluorescence data set representative of said sample. In one embodiment, this first detector may be selected from the group consisting of: a focal plane array detector, a CCD, an ICCD, a CMOS detector, and combinations thereof. In one embodiment, said fluorescence data set may comprise a hyperspectral fluorescence image. In another embodiment, said fluorescence data set may comprise at least one of: a fluorescence spectrum, a spatially accurate wavelength resolved fluorescence image, and combinations thereof.

A system of the present disclosure may further comprise a means for assessing a fluorescence data set to thereby determine at least one region of interest of said test biological sample. In one embodiment, a fluorescence data set may be assessed by visual inspection by a user. This embodiment may comprise a user locating areas of a fluorescence data set exhibiting characteristic fluorescence.

A system of the present disclosure may further comprise a second illumination source configured so as to illuminate a region of interest of said sample to thereby generate a second plurality of interacted photons. A second detector may be configured so as to detect said second plurality of interacted photons and generate at least one Raman data set representative of said region of interest. In one embodiment, this second detector may be selected from the group consisting of: a focal plane array detector, a CCD, an ICCD, a CMOS detector, and combinations thereof. In one embodiment, said Raman data set may comprise a hyperspectral Raman image. In another embodiment, said Raman data set may comprise at least one of: a Raman spectrum, a spatially accurate wavelength resolved Raman image, and combinations thereof.

A system may further comprise a means for assessing said Raman data set to thereby to thereby determine a diagnosis of said sample. In one embodiment, this diagnosis may comprise at least one of: a metabolic state of said sample, a clinical outcome of said sample, a disease progression of said sample, a disease state of said sample, and combinations thereof.

In one embodiment, a system of the present disclosure may also comprise at least one tunable filter configured so as to filter at least one of: said first plurality of interacted photons, said second plurality of interacted photons, and combinations thereof. This tunable filter may be selected from the group consisting of: a liquid crystal tunable filter, a multi-conjugate tunable filter, an acousto-optical tunable filter, a Lyot liquid crystal tunable filter, an Evans split-element liquid crystal tunable filter, a Solc liquid crystal tunable filter, a ferroelectric liquid crystal tunable filter, a Fabry Perot liquid crystal tunable filter, and combinations thereof.

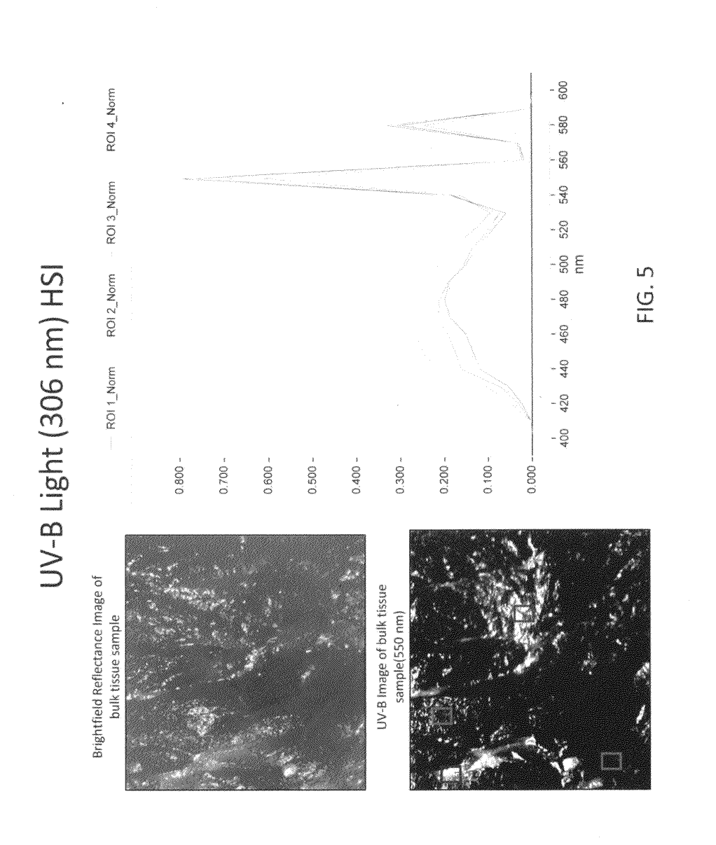

FIG. 5 is illustrative of the detection capabilities of the present disclosure. Fluorescence chemical image shows spatially different features in bulk tissue. Normalized image spectra represent these different regions. Different spectral features within these plots confirm chemical differences in the tissue. In one embodiment, using the system and method of the present disclosure, these regions may be further interrogated using Raman spectroscopic and/or imaging techniques.

The present disclosure also provides for methods and systems to use difference spectra, in particular Raman difference spectra, to diagnose samples. Based on the disease state and clinical outcome, a diagnosis of progressive or non-progressive disease may be provided. The use of difference spectra is more fully described in U.S. Patent Application Publication No. US2009/0040517, filed on Aug. 8, 2008, entitled “Raman Difference Spectra Based Disease Classification,” which is hereby incorporated by reference in its entirety.

While the disclosure has been described in detail in reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

What is claimed is:

1. A method comprising:
illuminating a test biological sample to thereby generate a first plurality of interacted photons;
detecting said first plurality of interacted photons to thereby generate at least one fluorescence data set representative of said sample;
analyzing said fluorescence data set to thereby determine at least one region of interest of said sample;
illuminating said region of interest to thereby generate a second plurality of interacted photons;
detecting said second plurality of interacted photons to thereby generate at least one Raman data set representative of said region of interest;
analyzing said Raman data set to thereby determine a diagnosis, wherein said diagnosis is selected from the group consisting of: a metabolic state of said sample, a clinical outcome of said sample, a disease progression of said sample, a disease state of said sample, and combinations thereof.

2. The method of claim 1 wherein said fluorescence data set comprises at least one hyperspectral fluorescence image.

3. The method of claim 1 wherein said fluorescence data set comprises at least one of: a fluorescence spectrum, a spatially accurate wavelength resolved fluorescence image, and combinations thereof.

4. The method of claim 1 wherein said Raman data set comprises at least one hyperspectral Raman image.

5. The method of claim 1 wherein said Raman data set comprises at least one of: a Raman spectrum, a spatially accurate wavelength resolved Raman image, and combinations thereof.

6. The method of claim 1 wherein said illuminating of said sample further comprises illuminating said sample using UV light.

7. The method of claim 1 wherein said illuminating of said region of interest further comprises illuminating said region of interest using substantially monochromatic light.

8. The method of claim 1 further comprising providing a reference database comprising a plurality of reference data sets, wherein each said reference data set is associated with at least one of: a known metabolic state, a known clinical outcome, a known disease progression, a known disease state, and combinations thereof.

9. The method of claim 1 wherein said analyzing of said fluorescence data set further comprises comparing said fluorescence data set to at least one reference data set.

10. The method of claim 1 wherein said analyzing of said Raman data set further comprises comparing said Raman data set to at least one reference data set.

11. The method of claim 1 wherein said analyzing of said fluorescence data set further comprises visually assessing said fluorescence data set to thereby identify said region of interest of said test biological sample, wherein said region of interest exhibits characteristic fluorescence.

12. The method of claim 9 wherein said comparing is achieved using a chemometric technique selected from the group consisting of: principle component analysis, partial least squares discriminate analysis, cosine correlation analysis, Euclidian distance analysis, k-means clustering, multivariate curve resolution, band t. entropy method, mahalanobis distance, adaptive subspace detector, spectral mixture resolution, and combinations thereof.

13. The method of claim 10 wherein said comparing is achieved using a chemometric technique selected from the group consisting of: principle component analysis, partial least squares discriminate analysis, cosine correlation analysis, Euclidian distance analysis, k-means clustering, multivariate curve resolution, band t. entropy method, mahalanobis distance, adaptive subspace detector, spectral mixture resolution, and combinations thereof.

14. The method of claim 1 wherein said test biological sample comprises at least one of: a kidney sample, a prostate sample, a breast sample, a pancreatic sample, a brain sample, a skin sample, an intestinal sample, a colon sample, a liver sample, a cardiac sample, a lung sample, an esophageal sample, a bladder sample, a blood sample, a urethral sample, an ovarian sample, a uterine sample, a testicular sample, a bone sample, a stomach sample, a tracheal sample, a tongue sample, a diaphragm sample, a nerve sample, and combinations thereof.

15. The method of claim 1 further comprising passing at least one of said first plurality of interacted photons and said second plurality of interacted photons through a tunable filter.

16. The method of claim 15 wherein said filter comprises a filter selected from the group consisting of: a liquid crystal tunable filter, a multi-conjugate tunable filter, an acousto-optical tunable filter, a Lyot liquid crystal tunable filter, an Evans split-element liquid crystal tunable filter, a Solc liquid crystal tunable filter, a ferroelectric liquid crystal tunable filter, a Fabry Perot liquid crystal tunable filter, and combinations thereof.

17. The method of claim 1 further comprising:
generating at least one of said fluorescence data set and said Raman data set at a data generation site;
transmitting at least one of said fluorescence data set and said Raman data set over a data communication network to an analysis center;
analyzing at least one of said fluorescence data set and said Raman data set at said analysis center to thereby determine a diagnosis, wherein said diagnosis is selected from the group consisting of: a metabolic state of said sample, a clinical outcome of said sample, a disease progression of said sample, a disease state of said sample, and combinations thereof; and
transferring said diagnosis to said data generation site via said data communication network.

18. A storage medium containing machine readable program code, which, when executed by a processor, causes said processor to perform the following:
illuminate a test biological sample to thereby generate a first plurality of interacted photons;
detect said first plurality of interacted photons to thereby generate at least one fluorescence data set representative of said sample;
analyze said fluorescence data set to thereby determine a region of interest of said sample;
illuminate said region of interest to thereby generate a second plurality of interacted photons;
detect said second plurality of interacted photons to thereby generate at least one Raman data set representative of said region of interest; and
analyze said Raman data set to thereby determine at least one of: a metabolic state of said sample, a clinical outcome of said sample, a disease progression of said sample, a disease state of said sample, and combinations thereof.

19. The storage medium of claim 18 wherein said machine readable program code, when executed by a processor, further causes said processor to pass at least one of said first plurality of interacted photons and said second plurality of interacted photons through a tunable filter.

20. The storage medium of claim 18 wherein said machine readable program code, when executed by a processor to analyze at least one of said fluorescence data set and said Raman data set, further causes said processor to:
compare at least one of said fluorescence data set and said Raman data set to at least one reference data set.

21. A system comprising:
a first illumination source configured so as to illuminate a test biological sample to thereby generate a first plurality of interacted photons;
a first detector configured so as to detect said first plurality of interacted photons and generate at least one fluorescence data set representative of said sample;
a means for analyzing said fluorescence data set to thereby determine a region of interest of said test biological sample; and
a second illumination source configured so as to illuminate a region of interest of said biological sample to thereby generate a second plurality of interacted photons;
a second detector configured so as to detect said second plurality of interacted photons and generate at least one Raman data set representative of said region of interest; and
a means for analyzing said Raman data set to thereby determine a diagnosis selected from the group consisting of: a metabolic state of said sample, a clinical outcome of said sample, a disease progression of said sample, a disease state of said sample, and combinations thereof.

22. The system of claim 21 further comprising at least one tunable filter configured so as to sequentially filter at least one of said first plurality of interacted photons and said second plurality of interacted photons into a plurality of predetermined wavelength bands.

23. The system of claim 22 wherein said tunable filter is selected from the group consisting of: a liquid crystal tunable filter, a multi-conjugate tunable filter, an acousto-optical tunable filter, a Lyot liquid crystal tunable filter, an Evans split-element liquid crystal tunable filter, a Solc liquid crystal tunable filter, a ferroelectric liquid crystal tunable filter, a Fabry Perot liquid crystal tunable filter, and combinations thereof.

24. The system of claim 21 wherein said first detector comprises a detector selected from the group consisting of: a focal plane array detector, a CCD, an ICCD, a CMOS detector, and combinations thereof.

25. The system of claim 21 wherein said second detector comprises a detector selected from the group consisting of: a focal plane array detector, a CCD, an ICCD, a CMOS detector, and combinations thereof.

26. The system of claim 21 wherein said fluorescence data set comprises a hyperspectral fluorescence image.

27. The system of claim 21 wherein said fluorescence data set comprises at least one of: a fluorescence spectrum, a spatially accurate wavelength resolved fluorescence image, and combinations thereof.

28. The system of claim 21 wherein said Raman data set comprises a hyperspectral Raman data set.

29. The system of claim 21 wherein said Raman data set comprises at least one of: a Raman spectrum, a spatially accurate wavelength resolved Raman image, and combinations thereof.
